(19) United States
(12) Patent Application Publication
(10) Pub. No.: US 2007/0196333 A1
(43) Pub. Date: Aug. 23, 2007

(54) Composition comprising mixtures of IFN-alpha subtypes

(76) Inventors: Fu-Yung Lin, Taipei (TW); Chih-Ping Yang, Taipei (TW); Shir-Ly Huang, Taipei (TW); Ching-Yuan Lee, Taipei (TW)

Correspondence Address: BAKER & MCKENZIE LLP, Pennzoil Place, South Tower, 711 Louisiana, Suite 3400, HOUSTON, TX, 77002-2716, US

(21) Appl. No.: 11/360,041

(22) Filed: Feb. 23, 2006

(51) Int. Cl. A61K 38/21; A61K 9/14; C12P 21/04; A61K 9/48; A61K 9/20

(52) U.S. Cl. 424/85.7; 424/451; 424/464; 424/46; 435/69.51

(57) ABSTRACT

A composition of human interferon-alpha (IFN-α) subtypes produced from human lymphoblastoid cells is disclosed. These purified IFN-α composition comprise higher specific activities and may be applied in the treatment of cancers, viruses, and immuno diseases.

### PRIOR RELATED APPLICATIONS

Not applicable.

### FEDERALLY SPONSORED RESEARCH STATEMENT

Not applicable.

### REFERENCE TO A SEQUENCE LISTING

Not applicable.

### FIELD OF THE INVENTION

This invention relates to an isolated protein composition, and more particularly to a composition of human interferon-alpha subtypes produced from a human lymphoblastoid cell.

### BACKGROUND OF THE INVENTION

The human interferon protein includes three main categories of IFN-α, IFN-β and IFN-γ. Among those subtypes, IFN-α has been identified as the most effective in therapeutic cancer formulations. Human IFN-α proteins are encoded by a multigene family comprising at least 13 genes clustered on human chromosome 9. The IFN-α subtypes have anywhere from 78% to 95% amino acid identity between the proteins (Henco, et al., 1985; Diaz, et al., 1996). Some of these α-interferon proteins have been shown to have antiviral, antigrowth and immunoregulatory activities. For example, eleven IFN-α subtypes have been identified from culture supernatants of Sendai virus-induced human leukocytes. Each IFN-α protein consists of 165 or 166 amino acid residues with molecular weights of between 18 and 27 kDa, depending on the degree of glycosylation and species of IFN-α (Tolo, et al., 2001; Nyman, et al., 1998; Zoon, et al., 1992).

The therapeutic efficacy of human IFN-α has been established for human diseases including cancers and viral infections. For example, partially purified leukocyte IFN-α which contained subtypes of IFN-α was used in clinical trials to treat viral infections (Stewart, et al., 1980). Additionally, U.S. Pat. No. 5,503,828 and U.S. Pat. No. 5,676,942 teach therapeutic human IFN-α products produced from human leukocyte cell lines. The disclosures of U.S. Pat. No. 5,503,828 and U.S. Pat. No. 5,676,942 are incorporated herein by reference.

Recombinant interferons (IFN-α2a, or IFNα-2b) have been used for the treatment of Condyloma acuminata, hepatitis B and C, AIDS related Kaposi's sarcoma, and regression of various malignancies. Recombinant interferons are currently in clinical trials for the treatment of SARS either alone or in combination with other antiviral agents (Cinatl, et al., 2003; Stroher, et al., 2004).

Recombinant IFNs are typically made using genetic engineering techniques, such as expression in E. coli. Specifically, IFN-α2 is the sole species in these products, such as INTRON A™ (IFN-α2b) by Schering Plough and ROFERON A™ (IFN-α2a) by Hoffman-La Roche. However, recombinantly produced interferons are composed of only a single human interferon subtype that has not been post-translationally modified or processed in vivo. Because recombinant interferons are not derived from a human cell line, they do not undergo processes such as glycosylation and their biological activities may be limited.

Interferons isolated from human cell lines have increased therapeutic efficacy when compared to recombinant interferons. For example, the alpha interferon derived from leukocyte cell lines can be used at a four times lower dosage to treat Condyloma than recombinant IFN-α.

Compositions of native human IFN-α subtypes also have several therapeutic benefits in comparison with single recombinant IFN-α subtypes. For example, patients treated with interferon compositions produced by native human cells have improved clearance of hepatitis B virus (HBV) over time than patients treated with the recombinant interferon (Lin, et al., 2004).

Natural sources of human interferon alpha include lymphoblastoid, leukocyte, Namalwa and peripheral blood leukocyte cell lines. IFN-α proteins purified from these cell lines can be referred to as native human interferons. Each native human interferon includes at least one interferon alpha subtype, each IFN-α subtype having a unique protein structure and biological activity dependent upon cell type, variant, and post-translational processing.

Increasing numbers of IFN-α preparations are now being used in patients and clinical trials for various indications. However, all have been characterized by a number of side effects. Side effects include flu-like symptoms such as fever, low blood cell counts, gastrointestinal disorders such as vomiting and diarrhea, renal disorders, pulmonary disorders, allergic reactions such as bronchospasm, anaphylaxis or skin rashes, hair loss, and infection. These side effects are reported in the product package inserts for all commercial IFN-α compositions.

For some treatments, the side effects of the interferon composition can have serious negative impacts on patients who must take significant dosages. To receive an effective dosage some patients must take large doses and/or dosages for long periods of time. The side effects produced by these large dosages can sometimes exceed the effects of the disease being treated.

Thus, there remains a need for a composition of native IFN-α compositions and mixtures thereof which have very low toxicity and high potency and which can also produce minimal side effects in patients undergoing interferon therapy, and a process of producing compositions of native IFN-α and mixtures thereof.

### SUMMARY OF THE INVENTION

A composition of purified native human IFN-α subtypes produced by human lymphoblastoid cells is described with improved anti-viral activities including increased potency in the treatment of viral diseases. In one embodiment of the invention, a native human IFN-α composition is purified from human lymphoblastoid cells which include at least one IFN-α subtype, wherein the molecular weight of the IFN-α subtype is approximately 19 to 27 kDa; and the antiviral activity of the IFN-α subtype is greater than 92 MIU/mg IFN. In another embodiment, the invention describes a native human IFN-α compositions purified from human lymphoblastoid cells which has IFN-α2, IFN-α2b, IFN-α2c, IFN-α4, IFN-α7, IFN-α8, IFN-α10, IFN-α16, IFN-α17, IFN-α21 and combinations thereof. Further the composition of native human IFN-α purified from human lymphoblastoid cells can have combinations of IFN-α2 and IFN-α8, IFN-α10 and IFN-α8, and IFN-α 17 and IFN-α8.

In still another aspect, a composition comprising IFN-α8, wherein said IFN-α8 is purified from a lymphoblastoid cell line. The composition can include at least one additional IFN-α subtype selected from the group consisting of IFN-α2, IFN-α2c, IFN-α2c, IFN-α4, IFN-α7, IFN-α10, IFN-α16, IFN-α17 and IFN-α21.

Additionally, a method of purifying native human IFN-α subtypes produced by lymphoblastoid is described where lymphoblastoid cells are cultured, IFN-α subtypes are separated from the culture media by affinity chromatography, and the IFN-α subtypes are separated by reverse-phase high-pressure liquid chromatography. Native IFN-α can be purified from lymphoblastoid cells, Narmalwa cells, or more specifically, the strain of cells at Taiwan deposit BCRC 960246 for Homo sapiens B lymphocyte (Namalwa) DB009, deposited Nov. 4, 2005.

In one aspect of the invention, a pharmaceutical composition includes comprising the above described native human IFN-α subtypes or native human IFN-α compositions purified by the method described above. The IFN-α subtype composition with improved antiviral activities may be in the following formulations: injection solution, injection powder for reconstitution, capsules, tablets, ointment, oral solution, syrups, inhalation powder or emulsions for therapeutic purposes.

The above described compositions can be use in mammals to treat viral infections, bacterial infections, fungal infections, and cancer. The compositions described may contain either a single IFN-α subtype or mixture of IFN-α subtypes. The composition may further include additional antiviral, antibacterial, antifungal, and anticancer agents including chemicals, proteins, or nucleotide treatments.

### DETAILED DESCRIPTION OF THE INVENTION

The term “interferon” as used herein, refers to any of a group of heat-stable soluble glycoproteins of low molecular weight that are produced by cells exposed to various stimuli, such as exposure to a virus, bacterium, fugus, parasite, neoplasm or other antigen.

“Type I” interferon family consists of 12 IFN-alpha subtypes, IFN-beta, and IFN-omega. Type I interferons described may be made by virus-induced lymphoblastoid cells or Narmalwa cell strains. A number of different interferon subtypes exist that are expressed by leukocytes, lymphoblastoids, Narmalwa cells, fibroblasts, and other cell types in response to viral infection, microbial infection or stimulation with double-stranded RNA.

“Interferon Alpha” or “IFN-α” is an interferon subtype expressed as from 14 genes on the short arm of chromosome 9 that code for these substances in humans. IFN-2A and -2B are protein products made by recombinant DNA techniques and are used as antineoplastic agents.

Antiviral activity is an ability to confer resistance to viral infection. Antiviral activity is measured using a WISH cell and encephalomyocarditis virus (EMCV) system. Briefly, WISH cells are seeded at 10⁴ cells per well in 96-well plates in DMEM medium supplemented with 10% FBS and preincubated for 6 hours. The cells are then treated with 2-fold dilutions of standard IFN-α (at the concentration of 500, 250, 125, 62.5, 31.3, and 15.6 IU/ml) and sample antiviral (around 200 IU/ml) IFN-α overnight. The cells are then challenged with EMCV until 75 to 95% cytopathic (CPE) is evident. Viable cells are measured with an XTT cell proliferation kit (“CELL PROLIFERATION KIT II (XTT)™” from ROCHE DIAGNOSTICS ™ Cat. No. 11-465-015-001), The sample activity is then calculated by the sample mean and calibrated to the standard IFN-α curve.

The term “isolated,” as used herein, refers to a nucleic acid or polypeptide removed from its native environment. An example of an isolated protein is a protein bound by a polyclonal antibody, rinsed to remove cellular debris, and utilized without further processing. Salt-cut protein preparations, size fractionated preparation, affinity-absorbed preparations, recombinant genes, recombinant protein, cell extracts from host cells that expressed the recombinant nucleic acid, media into which the recombinant protein has been secreted, and the like are also included. The term “isolated” is used because, for example, a protein bound to a solid support via another protein is at most 50% pure, yet isolated proteins are commonly and reliably used in the art.

“Purified,” as used herein refers to nucleic acids or polypeptides separated from their natural environment so that they are at least 95% of total nucleic acid or polypeptide in a given sample. Protein purity is assessed herein by one dimensional sodium dodecylsulfate polyacrylamide gel electrophoresis (SDS-PAGE) and silver staining. Nucleic acid purity is assessed by agarose gel and EtBr staining.

The term “substantially purified,” as used herein, refers to nucleic acid or protein sequences that are removed from their natural environment and are at least 75% pure. Preferably, at least 80, 85, or 90% purity is attained.

The phrases “nucleic acid” or “nucleic acid sequence,” as used herein, refers to polynucleotides, which may be gDNA, cDNA or RNA and which may be single-stranded or double-stranded. The term also includes peptide nucleic acid (PNA), or any chemically DNA-like or RNA-like material. “cDNA” refers to copy DNA made from mRNA that is naturally occurring in a cell. “gDNA” refers to genomic DNA. Combinations of the same are also possible (i.e., a recombinant nucleic acid that is part gDNA and part cDNA).

“Fragments” refers to those polypeptides (or nucleic acid sequences encoding such polypeptides) retaining antigenicity, a structural domain, or an enzymatic activity of the full-length protein. The “enzymatic activity” of the IFN-α protein is herein defined to be antiviral, antiproliferative and immunomodulating activities and influencing the metabolism, growth and differentiation of cells. “Structural domain” includes the interferon a/b domain which includes omega and tau. Helices A and C are also structural domains of the Type I IFN family.

A “variant” of IFNα polypeptides, as used herein, refers to an amino acid sequence that is altered by one or more amino acid residues. Such variations may be naturally occurring or synthetically prepared. Common variants include “conservative” changes, truncations, and domain removal or swapping with similar proteins. Guidance in determining which amino acid residues may be substituted, inserted, or deleted without abolishing biological or immunological activity may be found using computer programs well known in the art, for example, LASERGENE™ software, and comparison against the many known IFN-α1 genes.

The term “naturally occurring variant,” includes those protein or nucleic acid alleles that are naturally found in the population in question. The naturally occurring allelic variants may be point, splice, or other types of naturally occurring variations.

“High Stringency” refers to wash conditions of 0.2×SSC, 0.1% SDS at 65° C. “Medium stringency” refers to wash conditions of 0.2×SSC 0.1% SDS at 55° C.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova T A & Madden T L (1999) FEMS Microbiol. Lett. 174:247-250. The default parameters were used, except the filters are turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 11 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W wordsize [Integer] default=11 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at www.ncbi.nlm.nih.gov/BLAST/.

Recombinant IFN-α compositions do not possess. Lymphoblastoid IFN-α has significantly lower expression levels than recombinant IFN-α (to date), but its activity and specificity is much higher in inhibiting virus replication and the spread of malignant blastoma. Table 1 provides a listing of sequences taught herein.

TABLE 1
INTERFERON REFERENCE SEQUENCES^{a}

| | Descrip- | Amino | | | Amino |
|---|---|---|---|---|---|
| Acc # | tion | Acids | Acc # | Description | Acids |
| NP_076918 | IFN-α1 | 189 | NP_008831 | IFN-α13 | 189 |
| NP_000596 | IFN-α2 | 188 | NP_002163 | IFN-α14 | 189 |
| AAD13960 | IFN-α2a | 72 | NP_002164 | IFN-α16 | 189 |
| AAD13961 | IFN-α2b′ | 72 | NP_067091 | IFN-α17 | 189 |
| AAP20099 | IFN-α2b | 166 | NP_002166 | IFN-α21 | 189 |
| AAD13962 | IFN-α2c | 72 | NP_002167 | IFN-β1 | 187 |
| NP_066546 | IFN-α4 | 189 | NP_000591 | IFN-β2 | 212 |
| NP_002160 | IFN-α5 | 189 | NP_064509 | IFN-κ | 207 |
| NP_066282 | IFN-α6 | 189 | NP_000610 | IFN-γ | 166 |
| NP_066401 | IFN-α7 | 189 | NP_002168 | IFN-ω1 | 195 |
| NP_002161 | IFN-α8 | 189 | P37290 | IFN-δ1 | 195 |
| NP_002162 | IFN-α10 | 189 | NP_795372 | IFN-ε1 | 208 |

^{a}Reference sequences from GenBank ™ are presented as examples of the natural variation in interferons.

This invention provides a composition of human IFN-α and mixtures of human IFN-α produced by human lymphoblastoid cells, which is a cytokine regulatory factor overexpressing cell with the ability to produce at least 2-fold greater quantities of human IFN-α mixture as compared to other human lymphoblastoid cells (U.S. Pat. No. 6,159,712 and U.S. Pat. No. 6,489,144).

Due to increased cytokine production in this cell line, a lower cost for preparation and clinical testing of human IFN-α composition provided by this invention can be expected. It is contemplated that a composition of this invention may include a single IFN-α or a specific ratio of IFN-α subtypes. The compositions of this invention may be used alone as a pharmaceutical compositions, particularly for treatment of viral infections and cancer treatment. Furthermore, the compositions of this invention may be used as additives to known antiviral and antineoplastic pharmaceutical compositions to increase the potency and minimize the required dosages thereof. When added to another agent, the compositions of this invention may enable the use of pharmaceutical compositions containing smaller amounts than presently used. The ability to use smaller amounts of such known antiviral proteins or chemicals will thus minimize the severity of side effects which accompany conventional antiviral therapy. The compositions of this invention may be used in many formulations including, but not limited to: injection solution, injection powder for reconstitution, capsules, tablets, ointment, oral solution, syrups, inhalation powder or emulsions for therapeutic purposes.

This invention provides IFN-α composition with higher antiviral activity is characterized by antiviral activity assay. By the terms “higher antiviral activity” is meant that the antiviral composition of this invention has a greater antiviral activity than the known mixtures of IFN-α subtype or the commercial IFN-α2 product, such as Roferon A. The IFN-α compositions of this invention may be used alone as antiviral pharmaceutical compositions, or alternatively, may be used as additives to the known antiviral pharmaceuticals to enhance the antiviral potency and reduce the required dosages thereof. The compositions of this invention differ from naturally occurring and purified interferon mixtures, in that they are produced by the human lymphoblastoid cell, which is the U.S. patented cytokine regulatory factor overexpressing cell (U.S. Pat. Nos. 6,159,712 and 6,489,144). The IFN-α composition of this invention may be used in the presence of only one subtype, such as IFN-α8, or of a mixture of IFN-α2 and IFN-α8. The phrase “in the presence of only one subtype” is not limited to mean that a composition of this invention contains only one IFN-α subtype protein to the total weight of the pharmaceutical composition. In this invention, the phrase “in the presence of only one subtype” could be that a composition contains more than one IFN-α subtype selected from the group consisting of IFN-α2, IFN-α4, IFN-α7, IFN-α8, IFN-α10, IFN-α16, IFN-≢17 and IFN-α21. The selected IFN-α subtype composition, particularly to be used in the present invention can be defined by their amino acid sequences, molecular weight, function, and/or antigenicity. The IFN-α subtypes of one embodiment of this invention are isolated from a purified mixture of human IFN-α subtypes produced by the human lymphoblastoid cell.

The IFN-α useful in the antiviral compositions of this invention is at least one selected from the group consisting of IFN-α2, IFN-α4, IFN-α7, IFN-α8, IFN-α10, IFN-α16, IFN-α17 and IFN-α21. One embodiment of this invention is an antiviral composition that contains IFN-α8. Another embodiment of this invention includes at least one of IFN-α8 and one of IFN-α2. Still another embodiment of this invention comprises one IFN-α8 and a combination of one or more IFN-α proteins selected from IFN-α4, IFN-α7, IFN-α10, IFN-α16, IFN-α17 or IFN-α21. As stated above, one or any combination of these IFN-α compositions may provide a pharmaceutical composition having at least IFN-α8.

Further, this invention describes methods for purifying IFN-α subtype and combinations of IFN-α by reversed-phase high performance liquid chromatography (RP-HPLC) is disclosed and is described in detail in Example 1. The subtypes of IFN-α have slightly different hydrophobicities, which can be partially separated by an RP-HPLC column, eluting by organic solvents. The RP-HPLC columns used in the invention can be C4, C8 or C18, and the preferred column is C4. The organic solvents for RP-HPLC include but are not limited to methanol, ethanol, or acetonitrile (ACN). In one embodiment of this invention, the elution can be performed from 40 to 50% acetonitrile, and preferably is from 47.5 to 49.3% acetonitrile. The volume ratio of trifloroacetic acid (TFA) in the solvents can be from 0.5% to 2.0% and preferably is from 1.0% to 2.0%.

The physical and chemical properties of the IFN-α composition of this invention have been determined by several methods as described in detail in Examples 2 to 6. For example, the molecular weight of separated IFN-α subtype is determined by SDS-PAGE and the apparent molecular weights of the IFN-α subtypes are between 16 to 27 kilodaltons. Western-blot analysis of the IFN-α proteins under reducing conditions, demonstrates that purified protein bands are human interferons. The purity of the IFN-α composition from which the individual subtype or the IFN-α mixture useful in this invention is also measured by SDS-PAGE and by Western blot procedures, as discussed in detail in Example 4 below.

IFN-α subtypes were characterized by electrospray ionization quadrupole-time-of-flight mass spectrometry (ESI-Q-TOF MS) as described in detail in Example 3. The results show that at least 8 subtypes of IFN-α were purified including IFN-α2, IFN-α4, IFN-α7, IFN-α8, IFN-α10, IFN-α16, IFN-α17, IFN-α21.

Example 6 described the antiviral activity of an individual or a specific ratio of subtype of IFN-α determined by using human epithelial cell (WISH) against encephalomyocarditis virus (EMCV). The interferon unit in this measurement is defined as the reciprocal of the dilution at the 50% endpoint and is adjusted to the NIH reference standard (Ga 23-902-532). The antiviral specific activity of the IFN-α obtained range from 92 to 1268 MIU/mg IFN. The IFN amount is quantified by a commercial enzyme-linked immunosorbent assay (ELISA) kit from PBL, USA. It has been demonstrated that. IFN-α8 has the highest antiviral activity of about 1268 MIU/mg IFN, which is about 4.5 to 14-fold to the other IFN-α subtype in this invention. Furthermore, the composition comprising IFN-α2 and IFN-α8 in the molar ratio of 1:1 has the relatively higher antiviral activity, about 618 MIU/mg IFN, in comparison to the other composition in this invention. The composition mentioned above have the higher antiviral activities as compared to the commercial IFN-α2 product, Roferon A as described in Example 6.

### BRIEF DESCRIPTION OF THE DRAWINGS

FIG. 1—RP-HPLC Profile of Purified IFN-α Subtypes.

FIG. 2—SDS-PAGE of RP-HPLC Elution Peaks.

FIG. 3—Western-Blot of RP-HPLC Elution Peaks with LT-295 Monoclonal Antibody.

FIG. 4—Multiple Sequence Alignment of IFN-α Amino Acid Sequences.

### DESCRIPTION OF EMBODIMENTS OF THE INVENTION

This invention provides a composition of human IFN-α mixture produced by human lymphoblastoid cells, which is a cytokine regulatory factor overexpressing cell with the ability to produce at least 2-fold quantities of human IFN-α mixture as compared to other human lymphoblastoid cells (U.S. Pat. Nos. 6,159,712 and 6,489,144). Single IFN-α subtypes or a composition comprising a specific ratio of IFN-α subtypes with higher antiviral activities are described for use in treating viral infections, bacterial infections, fungal infections, or cancer. Compositions of IFN-α subtypes may be utilized in pharmaceutical formulations described in The Handbook of Pharmaceutical Excipients, including: injection solution, powder for reconstitution, capsules, tablets, ointments, oral solutions, syrups, inhalation powders, or emulsions for therapeutic purposes.

The following examples further illustrate the separation and characterization of IFN-α composition of the invention. These examples are not intended to limit the invention in any manner.

### EXAMPLE 1

### Lymphoblastoid Cell Growth

A Namalwa-derived (lymphoblastoid) cell line, DB009, is used in this example. Cells are grown in GM-11 medium which is composed of Pro293 (Cambrex, Md., U.S.A.), L-Glutamine, and MAXI-MAb (GTEC, CA, U.S.A.). When a cell count of 1.5×10⁶ to 2.0×10⁶ cells/ml is reached, the cells are diluted in the same medium to a concentration of 5×10⁵ cells/ml.

When cells are expanded to demand cell density (approximately 3.5−5.0×10⁶ cells/ml), they are treated with a priming reagent and incubated at 37° C. for 24 hours. Then Sendai virus is added and incubated for a further period in lower temperature (approximately 30-36° C.) at titer of 100 HA/10⁶ cells. The culture medium is separated from the DB009 cells as described below.

### EXAMPLE 2

### Purification of INF-A

All purification steps are performed at room temperature unless otherwise specified.

A. Crude Culture Medium Processing

Approximately 48 hours after Sendai virus induction, DB009 cells are removed by filtrating through 5 μm and 0.22 μm filter and the culture medium is collected. The filtered medium is stored in sterilized containers at 4° C. for further processing.

B. Affinity Purification

Human IFN-α is purified by affinity chromatography. A monoclonal antibody is coupled to CNBr activated Sepharose-4B and stored at 4° C. Approximately 0.2 mg of crude IFN-α filtrate is loaded per ml of affinity gel (with 2 mg coupled antibody). INF-α amount is determined by ELISA. The column is warmed up to room temperature from 4° C. before use, and is equilibrated with phosphate buffered saline (PBS). After sample loading, the column is washed with 5-column volumes of PBS containing 0.1% Tween 20 (PBST) followed by 20-column volumes of PBS containing 0.2 M KCl. The column is further washed with 5-column volumes of PBS. IFN-α is eluted from the affinity column with 50 mM citric acid buffer containing 0.3M NaCl (pH 2.0).

C. Acidic Incubation and pH Adjusting

The eluted interferon solution (about pH 2.0) from the monoclonal affinity column is incubated at 4° C. for 16˜20 hrs. The acidic incubation step is necessary to inactivate the Sendai virus. After acidic incubation, the solution is adjusted to pH 4.0 by mixing with 4-fold volume of 25 mM sodium acetate buffer (pH 4.0) containing 0.05% ZWITTERGENT® (CALBIOCHEM™) for further purification.

D. SP Sepharose Purification

The buffered IFN-α solution is loaded onto an SP SEPHAROSE® (SULFOPROPYL-SEPHAROSE®) column (SIGMA™). After sample loading, the column is washed first with 20-column volumes of “Buffer A” (25 mM sodium acetate, 0.05% ZWITTERGENT® (CALBIOCHEM™), pH 4.0), followed by 30-column volumes of mixture of 80% “Buffer A” and 20% “Buffer B” (25 mM sodium acetate, 1M NaCl, and 0.05% ZWITTERGENT® (CALBIOCHEM™), pH 4.0). The bound IFN-α is then eluted by 7-column volumes of solution containing of 50% “Buffer A” and 50% “Buffer B”. Finally, the SP column is rinsed with 10-column volumes of “Buffer B” to make remove residual IFN-α bound to the column.

E. G25 Desalting Column and Nanofiltration

A SEPHADEX G25® column (PHARMACIABIOTECH™) is equilibrated with 300 ml formulation buffer (10 mM Tris-HCl, 10 mM Glycine, and 145 mM NaCl; pH 7.0). The SP SEPHAROSE® eluate containing purified IFN-α is then loaded onto the G25 column followed by elution with formulation buffer. IFN-α fractions containing peak elution are aseptically pooled. Finally, in order to efficiently remove residual virus in the purified product, the purified IFN-α is filtered using a PLANOVA® 35N hollow fiber virus filter (PLANOVA™) of 35-nm pore size and stored at 4° C.

### EXAMPLE 3

### Separation of IFN-A Subtypes by RP-HPLC

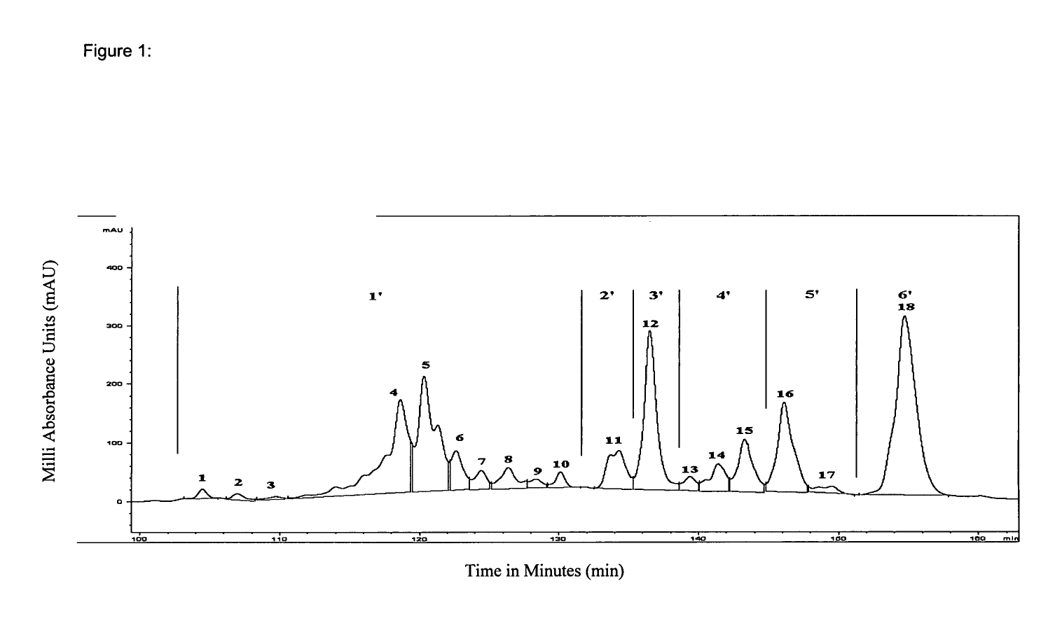

IFN-α subtypes are separated based on their relative hydrophobicity using RP-HPLC. Separation was achieved using an acetonitrile (ACN) concentration gradient. The RP-HPLC profile of IFN-α is obtained by loading approximately 15-20 μg of purified IFN-α onto an analytical 5 μm/300 A, 4.6×250 mm “Protein C4” column (GRACEVYDAC™, USA; Cat. No. 214TP54) and shown in FIG. 1. The linear elution gradient used for this C4 RP-HPLC is shown in Table 3 using the automated buffer gradients. Buffer A contains 0.15% trifluroacetic acid (TFA) in 100% H2O (v/v) and Buffer B contains 0.125% TFA in 100% ACN (v/v).

TABLE 3
YEAST IFN-A1 COMPARISON

| Time | Flow rate | % A | % B |
|---|---|---|---|
| 0 | 0.18 ml/min | 60 | 40 |
| 30 | 0.18 ml/min | 60 | 40 |
| 120 | 0.18 ml/min | 52.5 | 47.5 |
| 130 | 0.18 ml/min | 52.5 | 47.5 |
| 140 | 0.18 ml/min | 51.7 | 48.3 |
| 150 | 0.18 ml/min | 51.7 | 48.3 |
| 162 | 0.18 ml/min | 50.7 | 49.3 |
| 172 | 0.18 ml/min | 50.7 | 49.3 |
| 174 | 0.18 ml/min | 0 | 100 |
| 224 | 0.18 ml/min | 0 | 100 |
| 226 | 0.18 ml/min | 60 | 40 |
| 256 | 0.18 ml/min | 60 | 40 |

The purified IFN-α was fractionated into 18 peaks assigned as peaks 1 to 18. All of the peaks were collected separately. Some of these peaks may be pooled together to simplify analyses. Referring to FIG. 1, peaks 1 through 10 were combined and labeled as peak 1′; while peak 11 and 12 were pooled and labeled as peak 2′ and 3′, respectively; peaks 13 through 15 were pooled together and labeled as peak 4′; while peaks 16 and 17 were combined and labeled peak 5′; finally, peak 18 is labeled as peak 6′. The pooled protein peaks were lyophylized by SPEEDVAC® (Savant) and stored for later use. Lyophylized protein could be reconstituted in 50 mM phosphate buffer at pH 7.0 for subsequent analyses as needed.

### EXAMPLE 4

### One-Dimensional SDS-PAGE

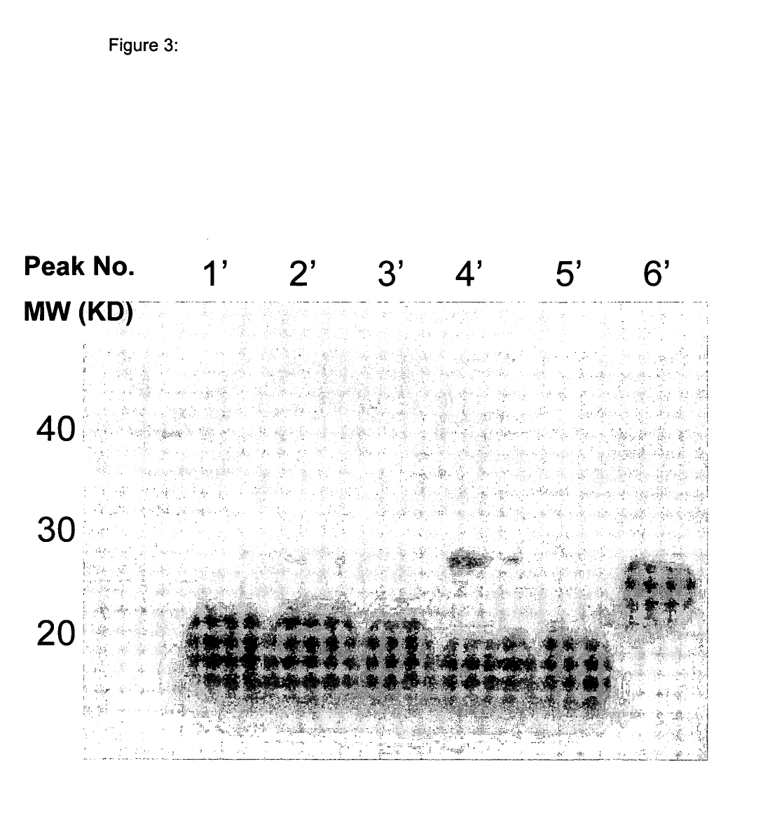

FIG. 3 Western blot of with LT-295 monoclonal antibody specific for IFN-α subtypes.

SDS-PAGE analyses are performed by using the procedures disclosed in Cleveland, et al. 1977. The IFN-α faction obtained from example 1 is analyzed in 16% SDS-PAGE under reducing condition. The protein bands are visualized by silver staining. A Western-blot of a duplicate SDS-PAGE is electroblotted and immuno-stained with LT-295 monoclonal antibody (from PBL, USA) specific to human IFN-α.

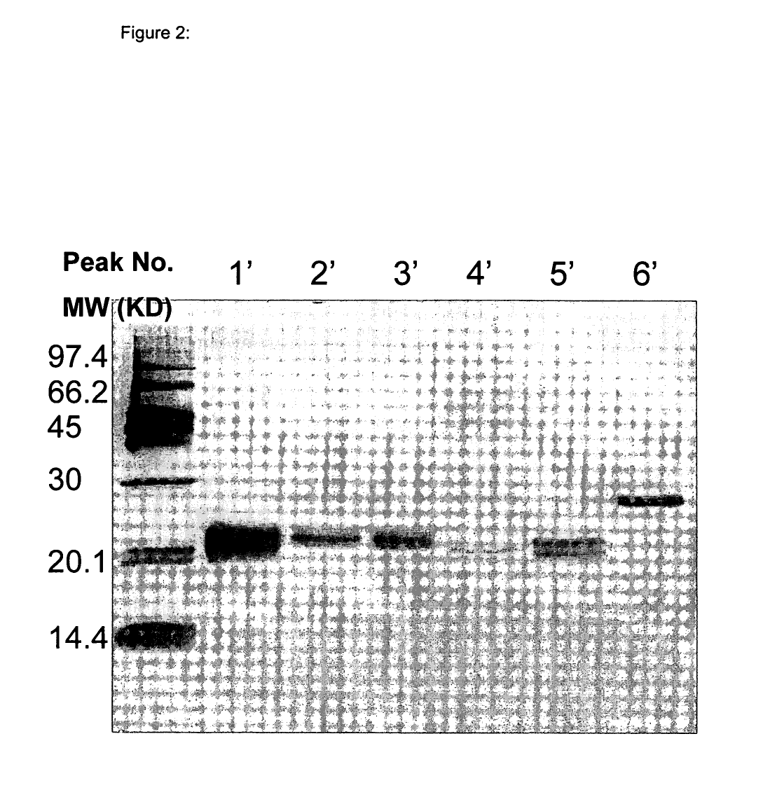

The data shown in FIGS. 2 and 3 demonstrate heterogeneity (i.e., more than one protein band) in some of the separated peaks. The relative molecular weights are calculated by using the molecular weight markers. The molecular weight markers (AMERSHAM™) are phosphorylase b (97.4 kD), serum albumin (66.2 kD), ovalbumin (45 kD), carbonic anhydrase (30 kD), trypsin inhibitor (20.1 kD), lysozyme (14.4 kD). The results from Western-blot analysis under reducing condition show that every protein band detectable by silver staining is recognized by the LT-295 monoclonal antibody. This demonstrates that all protein bands detected with the SDS-PAGE gel (FIG. 2) are IFN-α subtypes. Western blot analysis of the purified IFN-α compositions indicates that the purified native IFN-α subtypes have an less than 1% unfractionated interferon impurities. Subsequent fractionation on RP-HPLC shows no detectable impurity in any of the peaks.

### EXAMPLE 5

### Mass Spectrometry Characterization

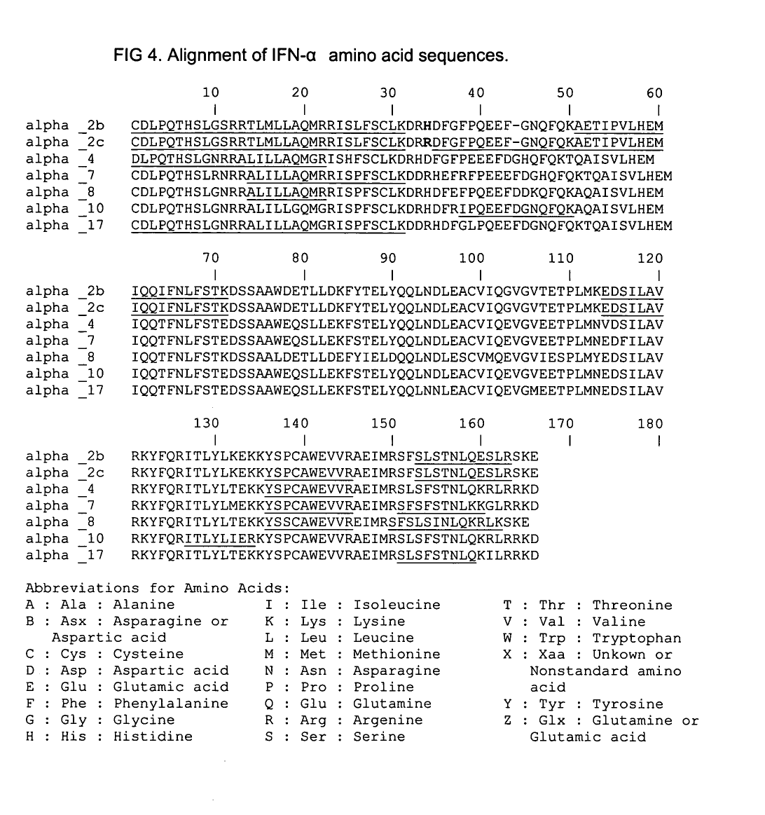

These data reveal that peak 1′ is IFN-α2; peak 2′ is IFN-α4; peak 3′ is IFN-α10; peak 4′ is IFN-α17 and IFN-α8; peak 5′ is IFN-α7; peak 6′ is IFN-α8, as listed in Table 3. It is noted that IFN-α2 may contain two subtypes, IFN-α2b and IFN-α2c. FIG. 4 demonstrates that some IFN-α2c is obtained through these purification techniques and IFN-α2b may be present as well. There is only one amino acid difference at position 33 in the amino acid sequences of IFN-α2b and IFN-α2c. Only IFN-α2c which contains the protease site from residues 34 to 49 can be detected by MS.

TABLE 4
IDENTIFICATION OF RP-HPLC ELUTION PEAKS

| Peak | IFN-α subtype |
|---|---|
| 1′ | IFN-α2 (IFN-α2c/b) |
| 2′ | IFN-α4 |
| 3′ | IFN-α10 |
| 4′ | IFN-α17 and IFN-α8 |
| | (minor) |
| 5′ | IFN-α7 |
| 6′ | IFN-α8 |

### EXAMPLE 6

### Antiviral Assay

The antiviral assay is performed by using WISH cells with EMCV. The interferon is serially diluted in 96-well plates, followed by addition of 10,000 cells per well. After incubation overnight, the cells are infected with EMCV, followed by an additional overnight incubation. Cytopathic effect (CPE) is checked microscopically on virus control, cell control and cells that received standard interferon doses. Cells are treated with “CELL PROLIFERATION KIT II (XTT)™” from ROCHE DIAGNOSTICS™ (Cat. No. 11-465-015-001) to be detected by colorimetric detector, when the wells containing standard interferon shows proper CPE. For all samples, 50% CPE was calculated. The interferon titer is then obtained by comparing with standardized NIH interferon reference (Ga 23-902-532). The interferon is quantified by “HUMAN INTERFERON ALPHA ELISA KIT™” (PBL-BIOMEDICAL LABORATROIES™, NJ. Cat. No. 41105-1). The results (Table 5) demonstrate that peak 6′ (IFN-α8) contains the highest specific activity and this is about 2.5 to 14 fold higher than that of the other IFN-α subtypes. The commercial IFN-α2a product, Roferon A, is employed as an index with a specific activity measured at about 250 MIU/mg IFN. For easy reference, absolute concentrations of 2 ng/ml IFN-α2 corresponds to 1000 IU/ml based on antiviral activity.
1 MIU=10⁶ IU=1 million inhibitory units (MIU) of specific activity.

The specific biological activity data presented in Tables 4 and 5 are in terms of the number of biological units per mg of the IFN-α present. Furthermore, in Table 5 the composition of IFN-α8 and IFN-α2 with a molar ratio of 1:1 has the highest specific activity as compared to the other mixture composition, and has about 1030 MIU/mg of IFN.

TABLE 5
ANTIVIRAL ACTIVITY OF IFN-A SUBTYPES

| | Peak | | | | | |
|---|---|---|---|---|---|---|
| | 1′ | 2′ | 3′ | 4′ | 5′ | 6′ |
| IFN-α | IFN-α2 | IFN-α4 | IFN-α10 | IFN-α17 | IFN-α7 | IFN-α8 |
| subtype | | | | IFN-α8 | | |
| Antiviral | 283 | 92 | 670 | 422 | 280 | 1268 |
| activity | | | | | | |
| [MIU/mg | | | | | | |
| IFN] | | | | | | |

TABLE 6
ANTIVIRAL ACTIVITY OF MIXED IFN-A SUBTYPE

| | Ratio of IFN-α subtype (Molar ratio) | | |
|---|---|---|---|
| | IFN-α2:IFN-α8 | IFN-α10:IFN-α8 | IFN-α17:IFN-α8 |
| | (1:1) | (1:1) | (1:1) |
| Antiviral | 1030 | 850 | 723 |
| activity | | | |
| [MIU/mg IFN] | | | |

As a consequence, the IFN-α compositions with improved anti-viral activities may be used alone or in combination as a pharmaceutical composition, particularly for use against viral diseases. In alternative, the compositions of this invention may be used as additives to the known agents so as to enhance the potency and reduce the required dosages thereof. The IFN-α composition may be utilized in, but is not limited to, the following formulations: injection solution, powder for reconstitution, capsules, tablets, ointment, oral solution, syrups, or emulsions for therapeutic purposes.

Realizations in accordance with the present invention have been described in the context of particular embodiments. These embodiments are meant to be illustrative and not limiting. Many variations, modifications, additions, and improvements are possible. Accordingly, plural instances may be provided for components described herein as a single instance. Additionally, structures and functionality presented as discrete components in the exemplary configurations may be implemented as a combined structure or component. These and other variations, modifications, additions, and improvements may fall within the scope of the invention as defined in the claims that follow.

### REFERENCES

All citations are hereby expressly incorporated by reference and are relisted here for convenience:
1. U.S. Pat. No. 5,503,828, Testa, et al. “Alpha interferon composition and method for its production from human peripheral blood leukocytes”. 1996.
2. U.S. Pat. No. 5,676,942, Testa, D. et al. “Composition containing human alpha interferon species proteins and method for use thereof”. 1997.
3. U.S. Pat. No. 6,159,712, Lau, S. A. “Methods for enhancing the production of interferon in cell culture” 2000.
4. U.S. Pat. No. 6,489,144, Lau, S. A. “Methods for enhancing the production of interferon in cell culture”. 2002.
5. Cinatl, et al., “Treatment of SARS with human interferons”, Lancet 362:293-4 (2003).
6. Diaz, et al., “Nomenclature of the human interferon genes”, J. Interferon Cytokine Res. 16: 179-80 (1996).
7. Evinger, M., et al, “Antiproliferative and antiviral activities of human leukocyte interferons”, Arch. Biochem. Biophys. 210, 319-29 (1981).
8. Fish, E. N., et al. “Human leukocyte interferon subtypes have different antiproliferative and antiviral activities on human cells”, Biophys. Res. Commun. 112: 537-46 (1983).
9. Foster, G. R., et al. “Different relative activities of human cell-derived interferon-alpha subtypes: IFN-alpha 8 has very high antiviral potency”, J. Interferon Cytokine Res. 16:1027-33 (1996).
10. Gewert, D., et al. “Inhibition of cell proliferation by interferons: 1. Effect on cell division and DNA synthesis in human lymphoblast cells”, Eur. J. Biochem. 139: 619-25 (1984).
11. Goodbourn, S., et al., “Interferon: cell signaling, immune modulation, antiviral responses and virus countermeasures”, J. General. Virol. 81:2341-64 (2000).
12. Henco, K., et al., “Structure relationship of human interferon gene and pseudogenes”, J. Mol. Biol. 185: 227-60 (1985).
13. Lin S. M., et al., “Comparison of long-term effects of lymphoblastoid interferon alpha and recombinant interferon alpha-2a therapy in patients with chronic hepatitis B”, J. Viral Hepat. 11:349-57 (2004).
14. Meager, A., et al., “Establishment of new and replacement World Health Organization International Biological Standards for human interferon alpha and omega”, J. Immunol. Method. 257:17-33 (2001).
15. Nyman, T. A., et al., “Identification of nine interferon-α subtypes produced by Sendai virus induced human peripheral blood leucocytes,” Biochem. J. 329:295-302 (1998).
16. Rowe, et al. “Handbook of Pharmaceutical Excipients” Pharmaceutical Press/APA, (2003).
17. Samuel, C. E., “Antiviral actions of interferons”, Clinical Microbiol. Rev. 14:778-809 (2001).
18. Stroher, U., et al., “Severe acute respiratory syndrome—related Coronavirus is inhibited by interferon-α. J”, Infectious Diseases 189:1164-7 (2004).
19. Stewart, et al., “Comparisons of several biological and physicochemical properties of human leukocyte interferons produced my human leukocytes and by E. coli,” Gene 11:181-6 (1980).
20. Thomson, A. W., et al., Cytokine Handbook, 4th edition, p 557.
21. Tolo, H., et al., “Development of a highly purified multicomponent leukocyte IFN-α product”, J. Interferon Cytokine Res. 21:913-20 (2001).
22. Yanai, Y., et al., “Analysis of the antiviral activities of nature IFN-α preparation and their subtype compositions”, J. Interferon Cytokine Res. 21: 835-41 (2001).
23. Zoon, K. C. et al., “Purification and characterization of multiple components of human lymphoblastoid interferon-α”, J. Biol. Chem. 267: 15210-6 (1992).

What is claimed is:

1. A composition comprising native human interferon alpha (IFN-α) wherein said native human IFN-α is purified from human lymphoblastoid cells, wherein said native human IFN-α comprises at least one IFN-α subtype, wherein the molecular weight of said IFN-α subtype is approximately 19 to 27 kDa; and wherein said native human IFN-α subtype has an antiviral activity between about 90 and about 1300 MIU/mg IFN.

2. The composition of claim 1, wherein said native IFN-α comprises at least one IFN-α subtype selected from the group consisting of IFN-α2, IFN-α2b, IFN-α2c, IFN-α4, IFN-α7, IFN-α8, IFN-α10, IFN-α16, IFN-α17 and IFN-α21.

3. The composition of claim 1, wherein said native IFN-α comprises IFN-α8 and at least one IFN-α subtype selected from the group consisting of IFN-α2, IFN-α2b, IFN-α2c, IFN-α4, IFN-α7, IFN-α10, IFN-α16, IFN-α17 and IFN-α21.

4. The composition of claim 3, wherein said native IFN-α comprises IFN-α8 and IFN-α2, IFN-α8 and IFN-α10, or IFN-α8 and IFN-α17.

5. The composition of claim 1, comprising a pharmaceutical excipient selected from the group consisting of injection solution, powder for reconstitution, capsule, tablet, ointment, oral solution, syrup, inhalation powder and emulsion.

6. The composition of claim 1, wherein said native human IFN-α is purified from a human lymphoblastoid cell strain, Namalwa cell strain, or cell strain Accession No.: BCRC 960246.

7. A pharmaceutical composition comprising a native human IFN-α subtype, wherein said native human IFN-α subtype has a molecular weight between about 19,000 and about 27,000 daltons, and wherein said IFN-α has an antiviral activity between about 90 and about 1300 MIU/mg IFN.

8. The composition of claim 7, comprising a pharmaceutical excipient selected from the group consisting of injection solution, powder for reconstitution, capsule, tablet, ointment, oral solution, syrup, inhalation powder and emulsion.

9. The composition of claim 7, wherein the IFN-α subtype is selected from the group consisting of IFN-α2, IFN-α2c, IFN-α2c, IFN-α4, IFN-α7, IFN-α8, IFN-α10, IFN-α16, IFN-α17 and IFN-α21.

10. The composition of claim 7, wherein said composition comprises IFN-α8 and at least one IFN-α subtype selected from the group consisting of IFN-α2, IFN-α2b, IFN-α2c, IFN-α4, IFN-α7, IFN-α10, IFN-α16, IFN-α17 and IFN-α21.

11. The composition of claim 10, wherein said native IFN-α comprises IFN-α8 and IFN-α2, IFN-α8 and IFN-α10, or IFN-α8 and IFN-α17.

12. A method of producing native human IFN-α compositions comprising:
a. culturing human lymphoblastoid cells;
b. affinity chromatography; and
c. reverse-phase high-pressure liquid chromatography, wherein said native human IFN-α has an antiviral activity between about 90 and about 1300 MIU/mg IFN.

13. The method of claim 12, wherein said cells are selected from the group consisting of lymphoblastoid, Namalwa, and cell strain Accession No.: BCRC 960246.

14. The method of claim 12, wherein said cells are DB009 cells.

15. A composition comprising IFN-α8, wherein said IFN-α8 is purified from a lymphoblastoid cell line.

16. The composition of claim 15, further comprising at least one additional IFN-α subtype selected from the group consisting of IFN-α2, IFN-α2c, IFN-α2c, IFN-α4, IFN-α7, IFN-α10, IFN-α16, IFN-α17 and IFN-α21.

17. The composition of claim 15, wherein composition comprises IFN-α8 and IFN-α2, IFN-α8 and IFN-α10, or IFN-α8 and IFN-α17.

18. The composition of claim 15, further comprising IFN-α2 in a 1:1 ratio of IFN-α2 to IFN-α8.

19. The composition of claim 15, further comprising an antiviral protein.
